(19) United States
(12) Patent Application Publication
(10) Pub. No.: US 2013/0066308 A1
(43) Pub. Date: Mar. 14, 2013

(54) ABLATION-BASED THERAPY FOR BLADDER PATHOLOGIES

(71) Applicant: Jaime Landman, Corona del Mar, CA (US)

(72) Inventor: Jaime Landman, Corona del Mar, CA (US)

(21) Appl. No.: 13/601,886

(22) Filed: Aug. 31, 2012

Related U.S. Application Data
(60) Provisional application No. 61/529,647, filed on Aug. 31, 2011.

(51) Int. Cl. A61B 18/02; A61B 18/24; A61B 18/14

(52) U.S. Cl. CPC A61B 18/02; A61B 18/14; A61B 18/24
USPC 606/15; 606/41; 606/21

(57) ABSTRACT

An ablation balloon to be used for bladder diseases. The balloon is uniformly or selectively covered with physical mechanisms capable of treating various bladder pathologies such as electrodes, insulation material, material capable of local release of fluid; or material capable of protecting selected regions of the bladder; and sensors capable of sensing (when balloon surface is in contact with bladder wall, temperature, impedance, pressure; or any combination thereof); or any combination thereof

### CROSS REFERENCE TO RELATED APPLICATIONS

This application claims the benefit of Provisional Patent Application 61/529,647 filed on Aug. 31, 2011, which is incorporated by reference herein in its entirety.

### FIELD OF THE INVENTION

The invention relates to treatments for bladder pathologies. More particularly this invention relates to complete or selective intra-vesical bladder musculature denervation and cancer cell destruction using a balloon catheter.

### BACKGROUND

Urinary bladder pathology results from overactivity of the detrussor (bladder wall) musculature. Bladder pathology, including problems of bladder overactivity and possibly interstitial cystitis, are also thought to result from overactivity or improper activity of the innervations of the bladder.

Problems of bladder overactivity are typically resolved using medications to pharmacologically reduce over activity of the bladder's nerves. These medications, including anticholinergic medications, are used by a very large population of patients. The medications are effective in most, but not all cases. Additionally, the medications have significant systemic side effects which could be reduced by a localized treatment strategy.

### SUMMARY

Radiofrequency ablation has previously been described for nerve ablation in the renal arteries. The current device being described is not a catheter, but rather a balloon. Additionally, the idea is application of radiofrequency, laser, or cryoablation based ablation of the urinary bladder which has not been previously described for denervation of normal (non-aberrant) and/or aberrant nerve pathways. The concept of intravesical bladder denervation has also not been previously described.

In the first embodiment a device, for instance a catheter (passes through the urethra with ability to inflate a balloon in the lumen of the urinary bladder), is inserted into the bladder. The balloon can be a high volume but low pressure type such that it can conform to the shape of the bladder lumen and make contact with a large surface area within the bladder. The balloon contains radiofrequency electrode(s), laser, cryoablation technology, or other ablative energy sources which can be activated to ablate the bladder wall. The resulting limited ablation will be deployed such that there is partial or complete dennervation (removal of normal or non-aberrant and/or aberrant nerve endings) of the bladder. The balloon can then be deflated and removed.

In yet another iteration of the device, the balloon is similarly deployed. The balloon can be configured such that only selected portions of the bladder are ablated. For example, only the bladder trigone area may be ablated by selective placement OR deployment (on surface of balloon) of ablative mechanisms. The balloon can also be designed to have protective areas/elements for the ureteral orifices, for example (see FIG. 3c).

In one embodiment a method is provided to treat bladder pathologies comprising inserting a catheter into the bladder, wherein the catheter comprises a deflated balloon, and inflating said balloon, wherein said balloon comprises physical elements capable of ablating the pathologies.

In another embodiment of the method, the bladder pathologies are selected from a group consisting of bladder overactivity; interstitial cystitis, and bladder cancer.

In yet another embodiment of the method, the physical elements are selected from a group consisting of electrodes, insulation material, material capable of local release of fluid; or material capable of protecting selected regions of the bladder; and sensors capable of sensing (when balloon surface is in contact with bladder wall, temperature, impedance, pressure; or any combination thereof); or any combinations thereof

In yet another embodiment of the method, individuals in need of such a method are first identified according to conventional clinical practices.

In another embodiment, a device for treating bladder pathologies, comprising a catheter comprising a deflated balloon, wherein the balloon comprises physical elements capable of ablating said pathologies.

In another embodiment of the device, the balloon is a high volume—low pressure balloon.

In yet another embodiment of the device, the physical elements are found uniformly on the balloon or only on selective portions of said balloon.

In another embodiment of the device of paragraph, the physical elements are electrodes; membrane pads capable of releasing drugs/fluid; or materials capable of providing insulation to said bladder; or sensors (capable of sensing when balloon surface is in contact with bladder wall, bladder temperature, bladder impedance, bladder pressure; or any combination thereof); or any combinations thereof

Some of the advantages of this method/device over the prior ways of achieving the same purpose are as follows.
One time treatment rather than medication every day.
Will likely have no systemic side effects
Long-term a single treatment will be cost effective as medication will not be required
Will potentially be single outpatient treatment solution for bladder overactivity, possibly interstitial cystitis, and other forms of bladder pathology.

### BRIEF DESCRIPTION OF DRAWINGS

The devices and methods disclosed herein have applicability in bladder pathology such as bladder overactivity (the system actually has much broader scope for treatment of bladder pathology including overactivity, interstitial cystitis, bladder cancer (Transitional Cell Carcinoma of the bladder and Carcinoma in situ of the bladder) and other diseases). It may also have application for diseases of urinary voiding (urine passage) including lower urinary tract symptoms of benign prostatic hyperplasia.

FIG. 1: Shows one embodiment of the undeployed ablation balloon catheter. There is a drainage hole or holes 10 at the distal tip for drainage of the bladder or for instillation of materials to optimize ablation or contact with the bladder wall (eg. conductive irrigant for RF ablation). In one embodiment, the drainage hole can comprise of conductive material. The balloon 12 in FIG. 1 is undeployed so that the catheter can be inserted transurethrally. The balloon comprises ablative mechanisms/technologies. The ablation balloon catheter has a filling/emptying port 14 for filling and emptying of the ablation balloon. There is also a typical drainage hole 18 at the proximal end for filling or emptying of the bladder.

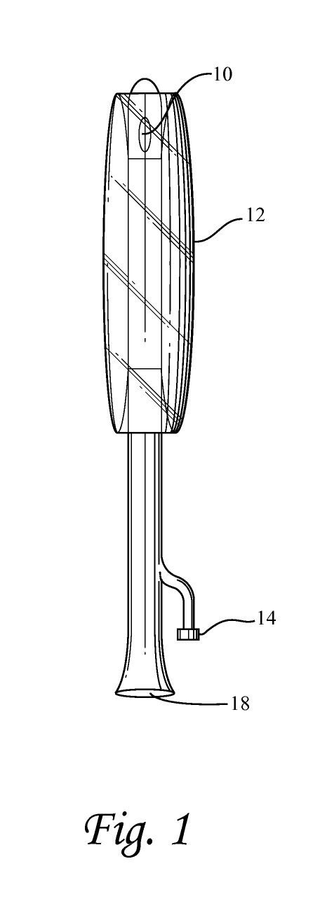

FIG. 2: Demonstrates in one embodiment, a filled high volume-low pressure balloon 12 which can make contact with a large portion of the inner wall of the bladder. The surface of the balloon 16 would contact ablative mechanism such as monopolar RF ablation, bipolar ablation, laser, ultrasound, drug releasing, cryoablation microwave or other ablative modalities which could be activated by user, and which could be limited by feedback mechanisms including temperature, impedance, and time based metrics. The user would activate the probe by passage of current through monopolar or bipolar electrodes on the outside of the balloon. In the case of cryoablation, the surface of the balloon, or parts of the surface, would be cooled to ablative temperatures by activating the cryoablation mechanisms known to those skilled in the art. The balloon can be inflated via a port, in one embodiment 14, either by a pump manually operated by the clinician/technician or via an automated system known in the art, or a combination of the two.

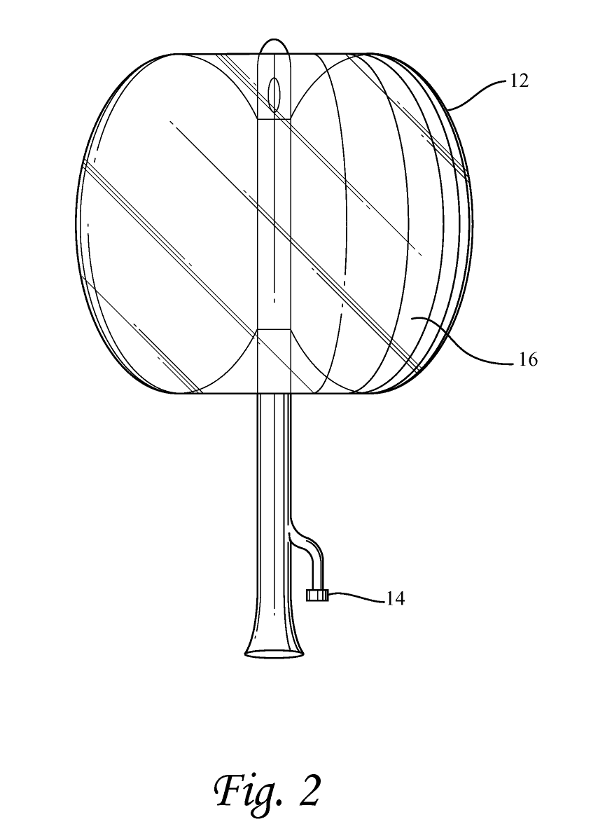

FIG. 3: Demonstrates three possible balloon embodiments. The first balloon (a) has ablative modality (as previously described) throughout the entire surface. The second balloon (b) has a selected portion of the surface of the balloon which has the ablative modality and can selectively ablate just one portion of the bladder (in figure just trigone area will be ablated). The third balloon (c) design demonstrates that selected portions of the bladder can be preserved from the ablation by insulation or heating/cooling technique. In this figure the area of the ureteral orifices is protected, but the technique could protect any portion of the bladder. Certainly, design permutations would potentially include combinations of these ideas. For example electrodes which cover entire surface of the bladder but could be activated separately (one part of the bladder at a time). Alternatively, ablation and protection could be incorporated into one balloon.

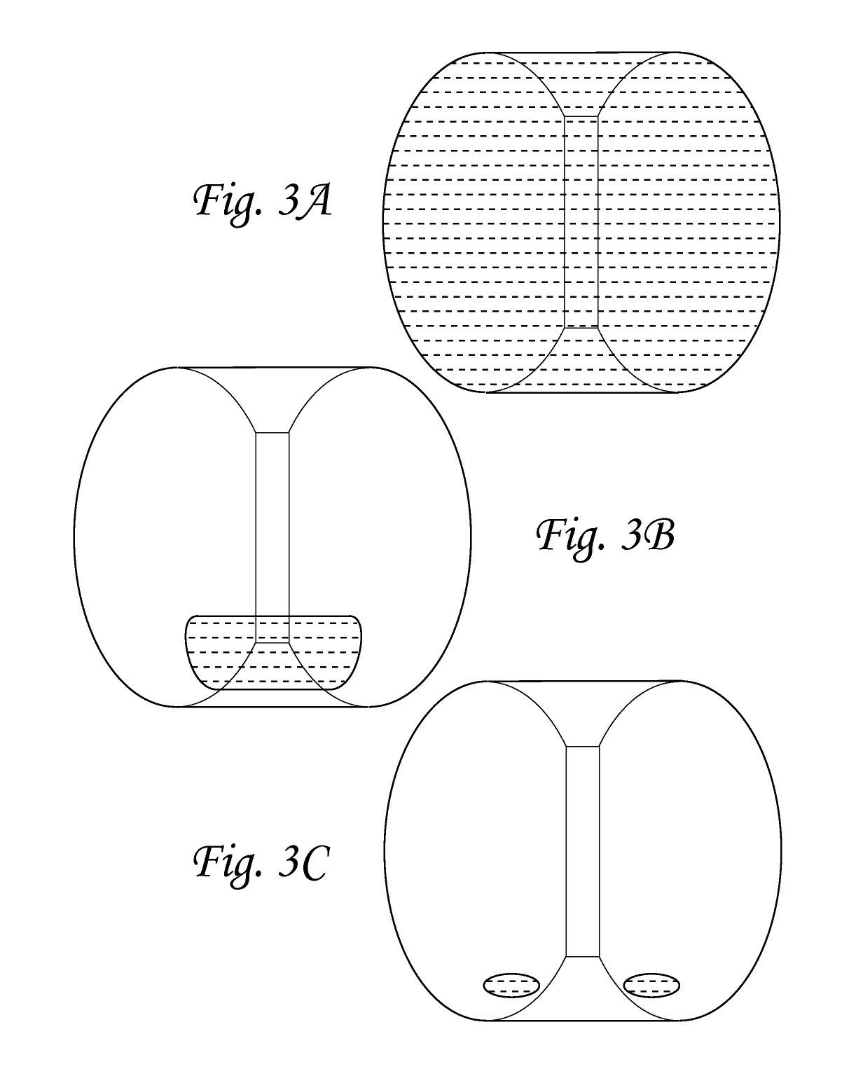

In other embodiments one or more balloons could be connected to the above mentioned catheters, each capable of delivering the same or different ablative mechanisms to the bladder either simultaneously or in a relay fashion.

### DETAILED DESCRIPTION

The invention is a concept and a technique for complete or selective bladder wall ablation for dennervation. The device is a balloon which can be deployed like a standard Foley catheter. The balloon is larger and is essentially a lower pressure high volume version of the standard Foley balloon so that it can fill the entire bladder wall making contact with the entire urothelial (inner lining) surface.

The term “catheter” is well known to those of skill in the art, including in reference to a hollow tube for insertion into a body cavity, duct, or vessel to allow the passage of fluids or to distend a passageway. The size of the catheter can be from either a range of 6 to 8 French up to larger catheters in the range of 24-26 French; in other embodiments between 12-30 French.

The term “ablation” as used in this application refers to destruction of tissue and/or nerves. The tissues or nerves ablated by the described method and device can be either normal/non-aberrant or aberrant.

The term “RF ablation” as used in this application refers to the use of electrodes to generate heat and destroy abnormal tissue. Here, in one embodiment, heat is generated locally by a high frequency, alternating current that flows from the exposed electrodes.

The term “monopolar RF ablation” as used in this application refers to the application of monopolar energy (the passage of energy from one or more electrode probes or something similar through local tissues and back to one or more electrodes at a distant location) for targeted destruction of tissue.

The term “bipolar ablation” as used in this application refers to the application of bipolar energy (the passage of electrical current within an instrument from one or more electrodes or something similar to one or more electrodes) for targeted destruction of tissue.

The term “cryoablation” as used in this application refers to the use of very cold thermal energy to lower the temperature of tissues and achieve tissue destruction. The temperatures used to achieve cryoablation are known to those of skill in the art.

The balloon will have radiofrequency electrodes, bipolar electrodes, laser or cryoablation technology on the outer surface so that the balloon can treat the bladder wall with ablative energy. The electrode distribution can be configured so that the entire inner surface of the bladder can be ablated.

For radiofrequency ablation, one or more electrodes are exposed to the tissue surface. Alternatively, the electrodes can be deployed on selected parts of the balloon so that only one portion of the bladder (eg. trigone, dome, etc.) will be ablated. The balloon can either be “hard wired” (i.e. have wires extending from the balloon and out to the electronic device/computer outside of the patient. Alternatively, the signal to and from the balloon can be wireless. In one embodiment, for radiofrequency ablation, a catheter comprises a balloon which contains one or more electrodes, inner fluid (i.e. saline), a thermocouple (to monitor the temperature within the balloon and tissue), and an agitator device (to constantly mix the saline so that the balloon remains at a constant temperature).

For cryoablation, in one embodiment the catheter can comprise of two balloons, an inner and an outer balloon. The inner balloon can be filled with nitrous oxide gas while the outer balloon can act as a protective covering to protect the tissue from coming into direct contact with the gas. The nitrous oxide gas will then ablate the tissue in contact with the balloon.

For laser ablation, in one embodiment the catheter can comprise of a balloon, which contains an endoscope, an optical fiber that delivers a laser beam. In another embodiment of the laser ablation device, the balloon also contains a small lumen to hold a coolant.

The balloon can also be partially insulated (or possibly have selected portions heated or cooled or insulation) to protect specific portions of the bladder including (among others) the ureteral orifices or the dome of the bladder. The technique can also be performed with prior deployment of ureteral catheters to protect the ureteral orifices. The dimensions of the balloon when inflated can vary from a smaller balloon (volume 5 to 10 cc) to larger balloons which engage the majority of the bladder surface (400-600 cc).

A device like catheter will be deployed into the bladder via the urethra. The balloon component of the device will be inflated and will make contact with the entire internal surface of the bladder wall. Radiofrequecy ablation, bipolar electrodes, laser or cryoablation technology on the surface of the balloon catheter will be deployed for either the entire inner surface, or for selected portions of the bladder wall (eg. trigone and bladder neck regions only). After deploying the device, a Foley catheter may or may not be deployed.

In any of the ablation technologies adopted, the balloon can also comprise of an endoscope and/or a deflectable sheath connected to the proximal end of the catheter.

Application of a high volume low pressure balloon that when inflated will make contact with entire bladder surface or a portion of the inner surface of the bladder. Denervation will allow treatment of many bladder diseases. Overactive bladder, one such disease, is currently typically treated with medications. With this device and technique, bladder overactivity and other diseases could be treated with single possible outpatient procedure.

The bladder is small when empty and consequently a large enough balloon of the present application will make contact with the majority of the inner surface. Histopathology of the bladder could also confirm areas of ablation. Sensors can also be placed uniformly or randomly on the surface of the balloon on the balloon surface capable of sending signals to the computer to generate computerized 3-D image of which portions of the balloon are in contact with the bladder.

Balloon activation can be designed such that it can be completely ablative throughout or can be designed to ablate only sections of the bladder (e.g trigone, bladder dome, lateral aspects of the bladder, etc.).

Once the inflated balloon makes contact with wall of bladder wall. RF energy, for instance, can then be applied. As will be apparent to those of skill in the art, energy, other than RF can also be applied.

The ablation balloon can also be used for bladder diseases that do not require dennervation, but rather need ablation of the surface. Superficial transitional cell carcinoma or carcinoma in situ of the bladder could be selectively ablated with the balloon.

1. A method to treat bladder pathologies comprising inserting a catheter into the bladder, wherein the catheter comprises a deflated balloon, and inflating said balloon, wherein said balloon comprises physical elements capable of ablating the pathologies.

2. The method of claim 1, wherein the physical elements results in denervation of normal or non-aberrant tissues/nerves and/or aberrant tissue/nerves.

3. The method of claim 1, wherein the bladder pathologies are selected from a group consisting of bladder overactivity; interstitial cystitis, and bladder cancer.

4. The method of claim 1, wherein said physical elements are selected from a group consisting of electrodes, fiber optics, cryogenic gas/liquid, insulation material, material capable of local release of fluid; or material capable of protecting selected regions of the bladder; and sensors capable of sensing when the balloon surface is in contact with bladder wall, temperature, impedance, pressure; or any combination thereof; or any combination thereof

5. The method of claim 4, wherein said electrodes are capable of heating or cooling said bladder.

6. The method of claim 1, wherein said balloon is a high volume-low pressure balloon.

7. The method of claim 1, wherein only selective portions of the balloon have said physical elements, and whereby the balloon is able to selectively treat specific regions of the bladder.

8. The method of claim 1, wherein said method causes musculature denervation or bladder cancer cell destruction.

9. A device for treating bladder pathologies, comprising a catheter comprising a deflated balloon, wherein the balloon comprises physical elements capable of ablating said pathologies.

10. The device of claim 9, further comprising a drainage hole located on the distal region of the catheter used for emptying or filling the bladder.

11. The device of claim 9, wherein the balloon is a high volume-low pressure balloon.

12. The balloon of claim 9, wherein said physical elements are found uniformly on the balloon or only on selective portions of said balloon.

13. The device of claim 9, wherein the physical elements are electrodes; membrane pads capable of releasing drugs/fluid; or materials capable of providing insulation to said bladder; or sensors capable of sensing when balloon surface is in contact with bladder wall, bladder temperature, bladder impedance, bladder pressure; or any combination thereof; or any combination thereof

14. The device of claim 9, wherein the physical elements are capable of generating laser beam, heat, cooling, drugs, sound waves, pressure, and/or mechanical vibration.
